(19) United States
(12) Patent Application Publication
(10) Pub. No.: US 2018/0214141 A1
(43) Pub. Date: Aug. 2, 2018

(54) Systems, Apparatuses, and Methods for Vessel Crossing and Closure

(71) Applicant: TransCaval Solutions, Inc., Miami, FL (US)

(72) Inventor: Max Pierre Mendez, Miami, FL (US)

(73) Assignee: TransCaval Solutions, Inc., Miami, FL (US)

(21) Appl. No.: 15/852,846

(22) Filed: Dec. 22, 2017

Related U.S. Application Data
(60) Provisional application No. 62/437,979, filed on Dec. 22, 2016.
(60) Provisional application No. 62/579,674, filed on Oct. 31, 2017.

(51) Int. Cl. A61B 17/00

(52) U.S. Cl. CPC A61B 17/0057; A61B 2017/00867; A61B 2017/00477; A61B 2017/00884; A61B 2017/00623; A61B 2017/00597; A61B 2017/00592; A61B 2017/0061; A61B 2017/00584

(57) ABSTRACT

An implant includes a collapsible tubular body, which, in an expanded configuration, extends from a first end to a second end centered along a longitudinal axis. The implant includes a hub coupled to the tubular body between the first and second ends. The hub is configured to removably connect to a deployment device. The deployment device is configured to manipulate and position the implant towards an implantation site in a vessel of a patient.

### CROSS-REFERENCE TO RELATED APPLICATIONS

This application claims priority under 35 U.S.C. 119(e) to U.S. Provisional Applications 62/437979, filed Dec. 22, 2016, and 62/579674, filed Oct. 31, 2017, the entire contents of all of which are incorporated herein by reference. Also, this application is related to U.S. application Ser. No. 15/060,960, filed Mar. 4, 2016, the entire contents of which are incorporated herein by reference.

### BACKGROUND

### 1. Field

The present disclosure relates to vascular and tissue closure devices, and, more specifically, to occlusion devices and methods for the closure of multi-vessel apertures, caused by venous-arterial access.

### 2. State of the Art

Complete percutaneous access into the arterial system up to the heart is desired. Limiting factors to this are arteries that do not facilitate current devices because of vessels that are atherosclerotic, tortuous, have a small diameter, are calcified, or have porcelain internal vascular walls. Anatomically parallel to the arterial system is the venous system, which does not typically have the same limiting properties. Percutaneous access into the venous system into the arterial system is advantageous and has been demonstrated and most impactful in caval-aortic procedures.

Transcaval access (TCA) is a new catheter technique that enables non-surgical introduction of large devices, such as transcatheter heart valves, into the abdominal aorta. TCA involves the creation of a conduit from the inferior vena cava (IVC) to the abdominal aorta (AA) by way of intravascular puncture and obturation of the resulting fistula. This fistula enables the introduction of large bore systems in a manner that bypasses existing arterial limitation. Upon completion of the TCA the caval-aortic fistula is closed with a commercial nitinol occluder device that is an off-label use. Such occluders have important limitations, such as residual bleeding and theorized potential complications. TCA has been performed successfully in dozens of patients to date.

However, in addition to closure limitations, the current method of caval-aortic crossing is limited in accuracy. Initially all patients undergoing TCA are assessed via computed tomography (CT) for anatomical features, as well as the identification of an ideal crossing zone and angle. This ideal crossing point is only assessed from within the AA, and it is defined as the least diseased, calcific, and obstructed portion of the AA. This ideal crossing point is also associated with a radiological angle, which is currently the sole means of synchrony between the CT and the fluoroscopic imaging during the procedure. The typical current method of crossing is as follows: Vascular access is gained in both the right femoral artery and right femoral vein. From within the artery an Amplatz GooseNeck® Snare (Covidien, Dublin, Ireland) is advanced into the arterial side, up until the snare is locate approximately at the site of ideal crossing. Through the venous side a 5 French Soft-Vu® Cobra Catheter (Angiodynamics, Inc., Latham, N.Y.) is advanced until parallel to the snare, it is then articulated so the distal end of the catheter is directed toward the snare. An ASAHI Confianza® 0.014″ Wire (Abbott Vascular, Santa Clara, Calif.) is blunted at the distal end, and an electrosurgery pencil is attached to the tip of the wire. This wire is then inserted into a 0.035″ PiggyBack° Wire Converter (Vascular Solutions, Inc. Minneapolis, Minn.) and this entire system is advanced within the Cobra Catheter. This system is then advanced out of the cobra catheter and the CT defined radiological angle is found. The system is rotated until facing the center of the parallel snare. This portion of the method is inaccurate, using an approximation for puncture based on a snare with a substantially larger diameter than the site of crossing. In addition, the IVC is a relatively dynamic vessel, directly influenced by the patient's respiration and fluid levels. This causes constant motion of the catheter system, and further decreases crossing accuracy. Once the system is placed in an approximated target zone, the wire system is engaged and the wire is rapidly punctured through the IVC wall into the interstitial space, and then through the AA wall. Common difficulties during this crossing are collision with calcific nodes within the AA, improper positioning, and puncture of additional structures. Upon puncture in the AA, the wire system is snared and exchanged for a stiff 0.035″ wire (of any configuration). The snared wire is then advanced upwards into the descending aorta, where it becomes an “S-like” guiderail for the large bore devices. Currently this method utilizes existing devices that are not purpose built for this type of procedure, which coupled with the lack of accuracy creates increased risk for an otherwise beneficial procedure.

### SUMMARY

According to one aspect, further details of which are described herein, a tubular implant, preferably embodied as an occulder, includes a tubular body covered partially by a hemostatic material and a hub extending at an angle from the tubular body. The implant is configured for deployment through a wall of a vessel to create a hemostatic seal around the opening in a vessel wall.

According to one embodiment, the tubular body includes a self-expanding (e.g., longitudinally and/or radially) tubular mesh that is formed of filaments. The filaments terminate at a side of the tubular mesh and are coupled at the hub. The tubular mesh is formed by an array of cells defined by an interwoven pattern. This tubular mesh may be formed from a metal such as stainless steel or a shape-set memory material such as Nitinol. The pattern array may also either be symmetrical about the central axis, or asymmetrical with a hemi-cylindrical region with a differing array of cells, or a woven structure.

The hub is movable relative to the tubular mesh. The filaments terminating at the hub extend at a non-zero angle with respect to an central longitudinal axis through the tubular body. The angle may be between 0 degrees and 180 degrees. The hub includes a connecting structure to removably the implant from a deployment device.

In one embodiment, a tether surrounds the tubular body or is otherwise coupled to the implant to retain the tubular body in a radially collapsed configuration during deployment of the implant completely through the wall of the vessel and to the deployment site within the vessel. The tether may be released from a proximal end of the deployment device to permit the tubular body of the implant to self-expand within the vessel. In one embodiment, the tether can be locked to a “spring” in the system that can allow for the tether to be displaced while maintaining tension without the tether being released.

The hemostatic or occlusive material is provided to the mesh at least about a circumferential portion suitable for creating a hemostatic seal about an opening in a vessel wall. The hemostatic or occlusive material may be formed of materials such as, but not limited to, PTFE and Dacron, which may be affixed to the outer or inner circumference of the mesh of the tubular body. In an embodiment, the hemostatic or occlusive material is a sealing fabric longer in width (circumferential dimension) than in length (axial or longitudinal dimension) and is sized to seal over the aperture of the punctured vessel. The sealing fabric is configured to promote clot formation, fill in vessel openings, and conform to irregular surfaces. Preferably, such a sealing fabric covers only a partial region of the mesh of the tubular body preventing any flow-through of fluids on only that covered region. Another occlusive material that can be used alone or in combination with the sealing fabric is a urethane coating coated on the tubular mesh. For example, according to one embodiment, a partial region of the tubular mesh may be dip coated in a liquid urethane to provide an even coating of urethane to the tubular mesh that extends within the cells of the mesh of the partial region. The urethane coating can provide additional hemostasis and facilitate sealing of the vessel puncture.

The purpose of the occlusive region is the selective and asymmetric hemostasis of solely the region of the vessel adjacent to the occlusive or hemostatic material. The uncovered region of the tubular body primarily functions as a stabilization and radial force-generating region only; it has no hemostatic or occlusive function. The uncovered section may also be formed from a horizontally discontinuous set of looped cell arrays, further reducing the profile and size of the structure.

According to one embodiment, the covered region may be further extended beyond the midline with the uncovered region removed, forming a “C-like” shape where there is a vertically discontinuous section that still retains enough radial force at the extremities to anchor in the lumen. For example, in at least one embodiment, the body of the implant may take the form of a portion of a cylinder, such as a hemi-cylinder.

According to one embodiment, an attachment site is located approximately at a central midpoint (between the longitudinal spaced ends of the mesh body) on the outer circumference of a hemi-cylindrical covered portion of the structure. This portion can have a circular metallic or metal-like hub affixed to it, providing central structural integrity and an attachment site for the mesh filaments. This hub structure may have a central self-closing opening for the passage of a guidewire or other device. In addition, the entire implant according to this embodiment may flex and/or pivot about the hub. A crimp may be provided to secure the hub over the filaments.

As noted hereinabove, the hub may also include a connecting structure for attachment of a distal end of a deployment device for delivering and deploying the implant. One connecting structure includes threads. Another connecting structure includes a bayonet locking structure. The hub may extend outwards providing a cylindrical or knob-like portion that provides additional anchoring and stability. This region has a pre-defined length and is to be seated within a natural or intentionally formed conduit such as a Caval-Aorto fistula. The hub may also be other shapes, such as disc-shaped or other flat-shaped. All shapes can apply a clamping force upon the vessel wall.

The tubular mesh is configured with a radially-expanding bias. The tubular mesh can be radially collapsed and constrained against its self-expanding bias. The aforementioned tether is configured to retain the tubular mesh in a collapsed configuration for delivery. The tether may extend around the circumference of the tubular mesh to constrain it in the collapsed configuration. The tether may extend around the exterior, or through one or more openings formed in the tubular body that may facilitate securing the tether relative to the implant during a deployment procedure. In one embodiment, the collapsed and tethered implant is configured to be loaded into a delivery catheter (i.e. vascular sheath) of a delivery system for deployment in a packaged configuration. In its compressed and packaged configuration, the implant will maintain a smaller profile than in the uncompressed, expanded configuration, allowing for smoother navigation and deployment of the implant in the vessel.

The delivery system also includes a shaft advanceable within the delivery catheter and that can be coupled at its distal end to the hub, such as via a threaded connection or complementary bayonet structure, and actuated, e.g., rotated or linearly actuated, via the proximal end of the delivery system. The shaft is preferably sufficiently flexible such that it can be advanced intravascularly without injury to the patient. The shaft is preferably sufficiently longitudinally stiff such that it can advance the tubular mesh through the delivery catheter and into the vessel without buckling. The shaft is preferably torsionally stiff such that it can transfer rotational force applied at the proximal end of the shaft to the distal end of the shaft, in as near a 1:1 ratio as practicable. Longitudinal displacement of the shaft relative to the delivery catheter permits manipulation of the implant relative to the target vessel. Rotation of the shaft relative to the implant results in release of the implant from the shaft.

In one embodiment, two ends of the tether extend through the delivery catheter and out of the proximal end of the delivery catheter. Once the implant is at the target location, one of the ends of the tether can be released and the other end of the tether can be withdrawn from the delivery catheter to cause the tether to be released from about the implant and withdrawn to permit the tubular body of the implant to expand within the vessel.

In one embodiment, the tether may have release knots or clips at some point along the implant that will allow for an end of the tether to be released to allow the frame to expand when the knots are untied. In this embodiment, the ends of the tether need not be located outside of the delivery catheter.

In one embodiment, the body of the implant retains its cylindrical shape while compressed within the delivery tube or catheter so that the outer circumference of the body is flush with the inner wall of the catheter. In this embodiment the tether extends in a plane perpendicular to the central axis of the body and the hub is pivoted so that it is parallel with the central longitudinal axis. In addition, the uncovered region is constricted circumferentially to lower the profile size further. Deployment of this embodiment of the implant relies on the advancement of the structure via the tether out of the delivery tube or catheter into the vessel lumen. As the implant leaves the catheter, the hub will gradually adjust its angle until the tubular body of the implant is concentric with the lumen of the vessel.

An additional packaged configuration of the implant is collapsed about the central hub of the implant while the tether is wrapped above the hub and above a center line (midpoint between the first and second ends of the body) of the body. Tension in the tether can be used to control the rotational bias of the body about the hub as the body expands upon its introduction into a vessel. Specifically, a user can control the tension in the tether to adjust the angle of the body pivoting about the hub, which is coupled to the deployment device. Therefore, by adjusting the tension in the tether, a user can control the angle of the body of the implant as the body expands inside the vessel.

Additionally, the tubular body of the implant is configured to expand to a tubular shape that is generally concentric within the vessel, but is at a diameter that is smaller than the vessel and then is able to expand by a user's control of the tether or an equivalent type method. Alternatively, a balloon expanded stent system can be used.

In one embodiment, the construction and pattern of a tubular mesh composed of woven wires can be arranged such that the volume required during expansion does not interfere with the vessel and, therefore, allows the structure to articulate and self-align concentrically within vessel. The woven wires can be arranged such that each strand has two free ends culminating into the central hub.

In one embodiment, a woven pattern of the tubular mesh can be created using a single wire to create the entire woven structure, with only the two ends of the wire within the hub. In yet another embodiment, the tubular mesh does not contain interwoven wires but contains a vertical array of angulating wires in a tubular arrangement. In a further embodiment, the tubular mesh is created from a single wall tube that is laser cut to create an array of diamond like patterns that are able to flatten to allow the overall tubular diameter to reduce or increase.

Additionally, individual loops of metallic wire can be used to support the hemostatic sealing structure against the vessel.

In an additional embodiment, a plurality of tubular mesh structures are linked together to seal multiple vessels in a sequential fashion.

Bioabsorbable materials can be used both for the tubular mesh structure and for the hemostatic and occlusive portion.

As an alternate to a tether that surrounds or is otherwise coupled to the implant to retain the implant in a radially collapsed configuration, a single clip or multiple clips can be used to maintain the implant in a radially collapsed configuration. The clip can be part of the delivery system and housed in proximity to the hub. The implant can be collapsed radially and loaded into the clips at points along its circumference. The user can actuate the handle to release the clips and permit the implant to self-expand within the vessel.

Also, while the hub structure is disclosed as having a central self-closing opening for the passage of a guidewire or other device, as an alternative a portion of the frame can be arranged such that it allows for the passage of devices once interrogated. A self-closing hinged or flexing gate can be used to facilitate device passage and immediate closing once retrieved.

Although the implant is illustrated and described herein as embodied in systems and methods of multi-vessel closure, it is, nevertheless, not intended to be limited to the details shown because various modifications and structural changes may be made therein without departing from the spirit and scope of this disclosure. By way of example, the structure of the individual occluders, alone or in combination with the deployment systems taught herein, can be used to seal and provide hemostasis at an aperture in a single tissue wall, including in a vessel, or in the wall of an organ, such as the heart, and more particularly, by way of example only, to treat atrial septal defects. Additionally, well-known elements of exemplary embodiments of the invention will not be described in detail or will be omitted so as not to obscure the relevant details of the invention.

Additional features are set forth in the detailed description that follows and may be apparent from the detailed description or may be learned by practice of exemplary embodiments. Other features and attendant benefits may be realized by any of the instrumentalities, methods, or combinations particularly pointed out in the present disclosure.

### BRIEF DESCRIPTION OF THE DRAWINGS

FIG. 1A shows an implant along section 1A-1A in FIG. 1B.

FIG. 1B is a perspective view of the implant of FIG. 1A.

FIG. 2A shows the implant of FIG. 2B along section 2A-2A in FIG. 2B.

FIG. 2B is a perspective view of the implant of FIG. 2A.

FIG. 2C shows a view of the implant of FIG. 2B along section 2C-2C in FIG. 2B.

FIG. 3 shows the implant of FIGS. 1A-1B in a collapsed delivery configuration in a delivery tube.

FIGS. 4A and 4B illustrate a stage in the implantation of the implant in the collapsed delivery configuration shown in FIG. 3.

FIG. 5 shows a distal portion of the implant being introduced into a vessel.

FIGS. 6 and 7 show the implant further advanced into the vessel than that shown in FIG. 5.

FIGS. 8 and 9 show the implant fully deployed into the vessel.

FIG. 10 illustrates an embodiment of a tool for forming a shape-set structure of an implant in accordance with an aspect of the disclosure.

FIG. 11 shows wires arranged on the tool in FIG. 10 connected to a rod.

FIG. 12 shows a shape-set structure in a collapsed configuration and surrounded and constrained by a tube.

FIG. 13 shows the shape-set structure of FIG. 12 in an expanded configuration.

FIG. 14 shows a view of the shape-set structure of FIG. 13 rotated ninety degrees from the position shown in FIG. 13.

FIGS. 15A to 15C show another embodiment of a shape-set structure made with an occluding material.

FIGS. 16A-16D illustrate sequential stages of deployment of the tubular woven structure of FIGS. 14 and 15A to 15C from a delivery tube into a clear tube, which is representative of a vessel.

FIGS. 17A-17D illustrate sequential stages of deployment of the tubular woven structure of FIGS. 15A to 15C from a delivery tube into a clear tube.

FIGS. 18A to 20 show another embodiment of an implant in accordance with an aspect of the disclosure.

FIG. 21 shows a deployment device coupled to the implant shown in FIGS. 18A to 20.

FIG. 22 shows a stage of advancing a guidewire through the inferior vena cava (IVC) and in a crossing manner and into the abdominal aorta (AA).

FIG. 23 shows an aperture in the abdominal aorta (AA) and a portion of the guidewire in the aperture.

FIG. 24 shows the introduction of a delivery catheter advanced into the abdominal aorta (AA) through the aperture.

FIGS. 25a-f show a sequence of operations for deploying the implant of FIGS. 18A to 20 in accordance with an aspect of the disclosure.

FIG. 26 shows an alternate embodiment of tethering an implant.

In the following detailed description, reference is made to the accompanying drawings which form a part hereof, and in which are shown by way of illustration embodiments that may be practiced. It is to be understood that other embodiments may be utilized and structural or logical changes may be made without departing from the scope.

### DETAILED DESCRIPTION

The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, and top/bottom. Such descriptions are merely used to facilitate the discussion and are not intended to restrict the application of disclosed embodiments. Various operations may be described as multiple discrete operations in tum, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

Herein various embodiments of the systems and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

As used herein, terms such as transcaval, TCA, TC, trans-caval, caval-aortic, aortocaval, aorto-caval, venous-arterial are the same. Terms such as aperture, opening, rent when used herein are the same. Terms such as tract, shunt, path when used herein are the same. Terms such as vessel, vessels, wall, walls, tissue, tissue wall, tissue walls, aortic vessel wall, venous vessel wall when used herein are the same.

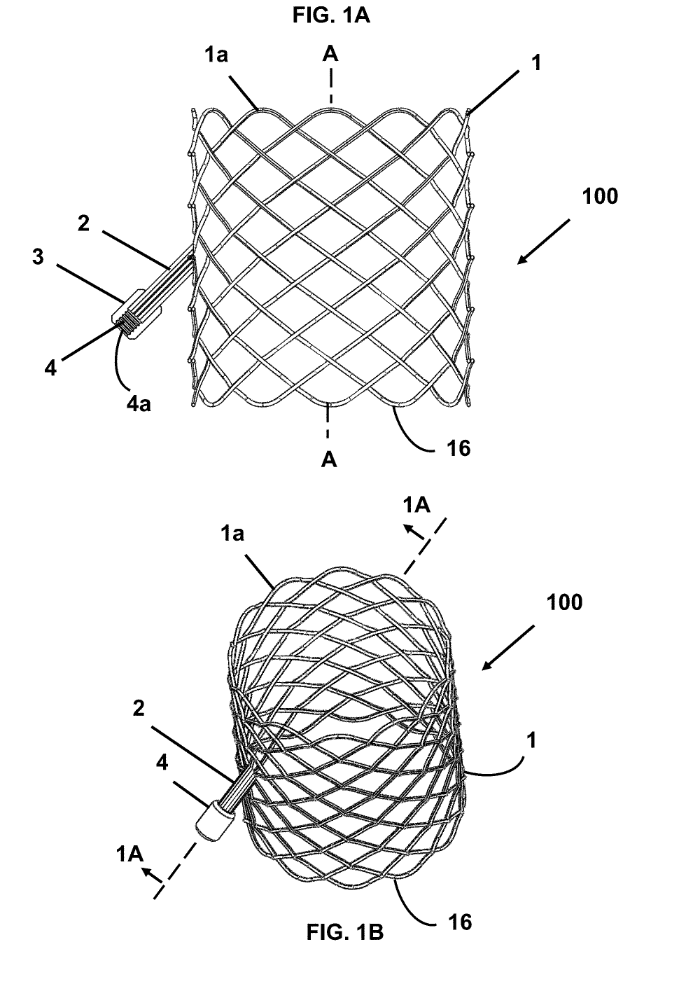

FIG. 1A and 1B show a tubular implant 100 configured as an occluder. The tubular implant 100 includes a tubular body 1 formed from a tubular mesh which is biased to expand radially outwardly when radially compressed. The mesh may be formed from a shape memory material and is able to undergo compression and expansion. The mesh may be formed of woven strands that extend at a single angled neck 2 where their ends 3 are crimped together. The implant includes a hub or port connected to the ends 3 of the strands of the neck 2. The hub is configured to connect to any control systems used for the manipulation and deployment of the implant 100. In the cutaway section shown in FIG. 1A, the hub 4 has a female threaded connection 4a for connecting to a mating threaded portion of a control system.

The tubular body 1 is shown in FIGS. 1A and 1B in an expanded configuration centered about a longitudinal axis A-A. The body 1 extends longitudinally from a first end 1a to a second end 1b.

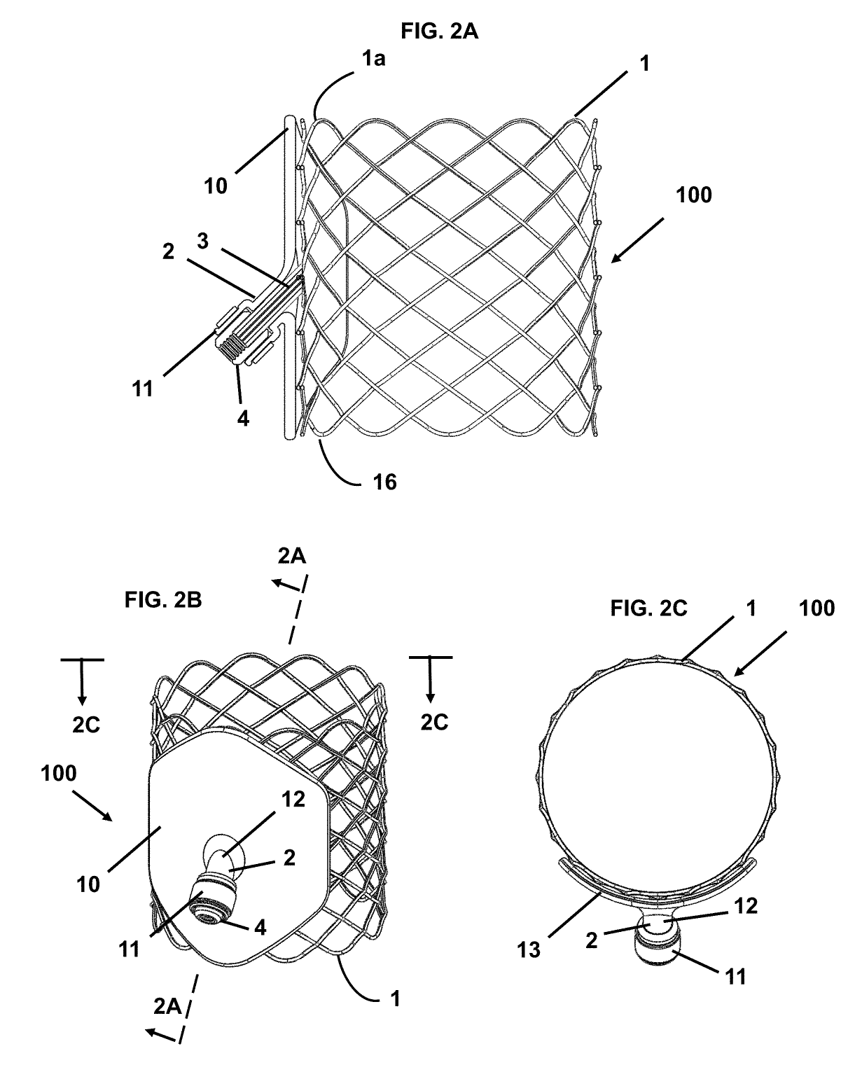

As shown in FIGS. 2A, 2B, and 2C, the implant 100 may include an occlusive material 10 that covers at least a portion of the tubular body 1. The occlusive material 10 is configured to occlude a vascular aperture through which the implant is deployed. The occlusive material 10 can be applied fully or partially around an outer or inner circumference of the tubular body 1. The occlusive material 10 is also drawn over the terminating ends 1a and 1b of the mesh body 1 and affixed to a crimp point 11 around the hub 4. This arrangement of the occlusive material 10 provides continuous coverage over the hub 4 and neck 2 which protrude at an angle from the side of the tubular body 1. As shown more clearly in FIGS. 2B and 2C, the hub 4 and neck 2 may also be independently covered by a separate yet affixed occlusive material 12 that is configured to prevent fraying or opening of the material 12 under manipulation. In the embodiments shown in FIGS. 2A, 2B, and 2C, the occlusive material 10 is affixed to an outer circumferential side of the mesh body 1 to create partial or full circumferential sealing of vascular conduit. Alternatively or additionally, the material 10 may also be affixed on an internal circumferential side of the mesh body 1.

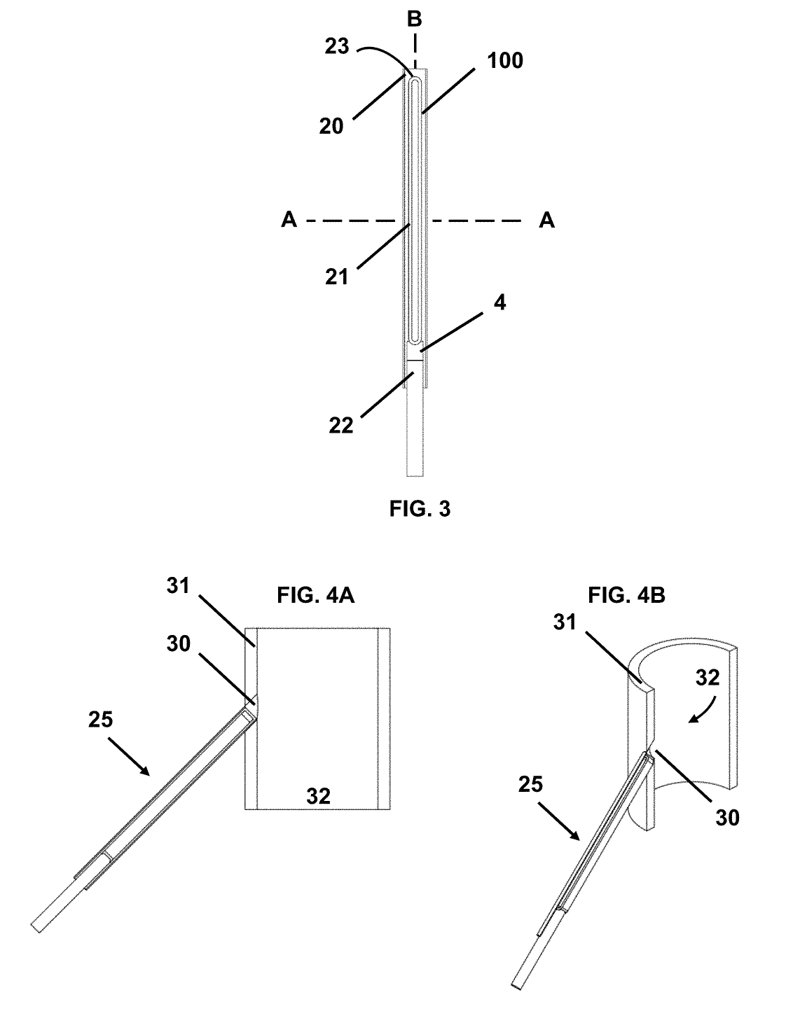

The implant 100 is configured to be compressed radially and axially for deployment into a substantially linear delivery configuration and loaded into a hollow deployment tube 20, forming an assembly 25 as shown, for example, in FIG. 3. In the linear delivery configuration shown in FIG. 3, the central longitudinal axis A-A of the body 1 is oriented transverse to a longitudinal axis B-B of the deployment tube 20. When the body 1 of the implant 100 is compressed, the body 1 can be retained in the compressed configuration by a tether 23 surrounding the body 1. When the implant 100 is in the tube 20, the hub 4 is coupled to a control shaft 22. The control shaft 22 allows the user to apply the force necessary to deploy the implant 100 from the collapsed configuration in the tube 20 to an expanded configuration outside of the tube 20.

As shown in FIGS. 4A and 4b the entire assembly 25 is configured to be inserted through a vascular aperture 30 in a vessel 32, which aperture 30 may be formed as a result of a vessel rupture, a vessel puncture, or a branched vessel that is located at a vessel wall 31 of the vessel 32.

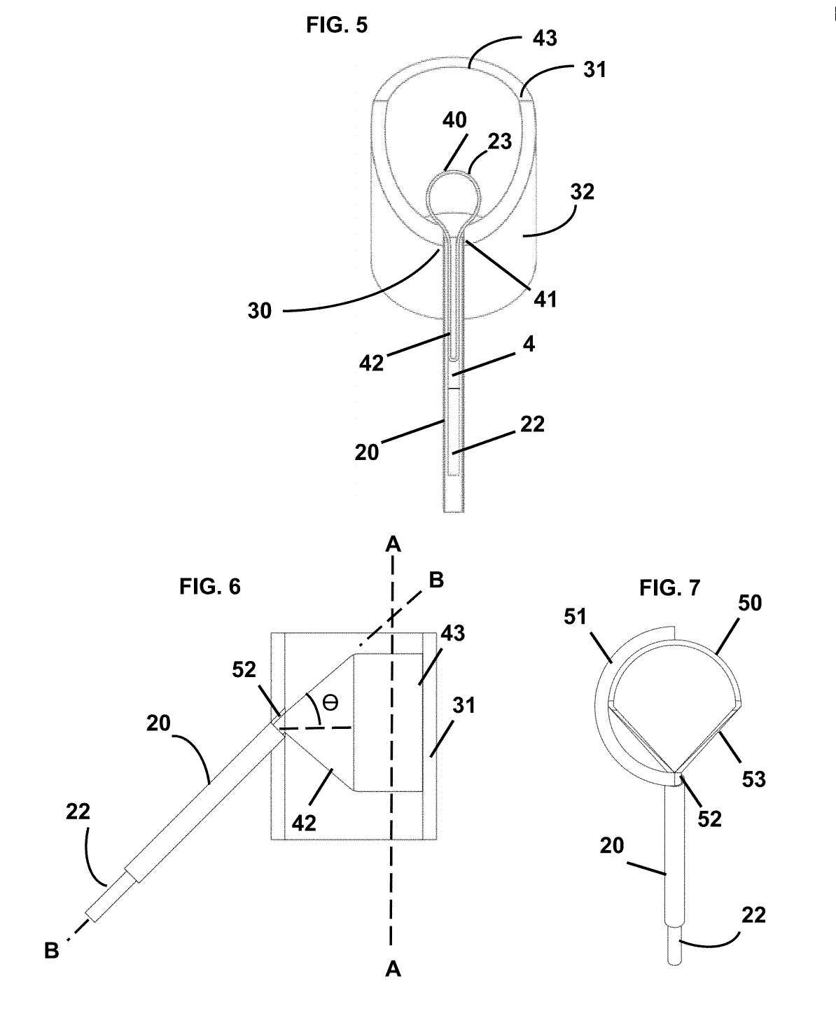

FIGS. 5 to 7B show stages of deploying the implant 100 into the vessel 32. A shown in FIG. 5, a distal portion 40 of the compressed body 1 is introduced into the vessel 32 towards a location 43 across from aperture 30 of the vessel 32. As the distal portion 40 is advanced further into the vessel 32, if the tether 23 is released, the distal portion 40 expands while a proximal portion 42 of the body 1 remains partially constricted within the delivery tube 20.

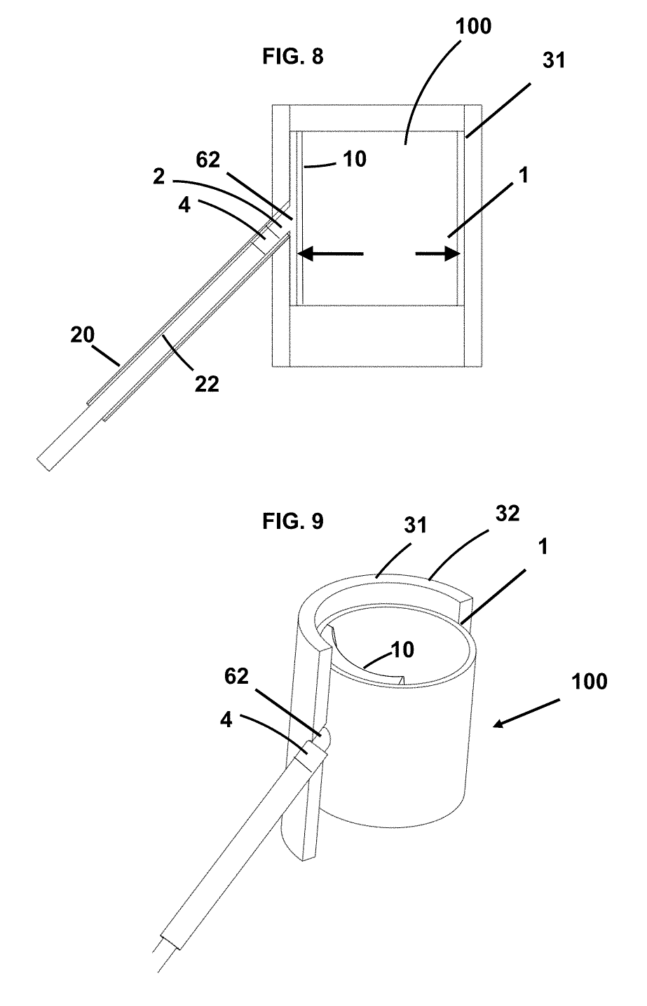

As shown in FIGS. 6 and 7, after further introduction of the distal portion 40 into the vessel 32, the distal portion 40 engages the location 43 of the vessel 32 across from aperture 30. Also, as shown in FIG. 6, the body 1 and its central axis A-A are shown rotated about an angle 0 with respect to the deployment axis B-B of the delivery tube 20 so that the body 1 aligns concentrically with respect to the vessel 32 and is properly apposed to the vessel wall 31. At this point in the deployment of the implant 100, a proximal end of the proximal portion 42 of the implant body 1 is still constricted by the delivery tube 20 and, therefore, only the portion of the proximal portion 42 in the vessel 32 is partially expanded. In one embodiment, the entire tubular body 1 is allowed to expand in the vessel 32 while maintaining a generally perpendicular relationship between the center axis B-B of the delivery tube and the center axis A-A of the structure, i.e., the angle δ is 0 degrees. Additionally, upon further introduction of the body 1 into the vessel 32, the body 1 is allowed to articulate in a passive, driven, or preset fashion to concentrically align itself with the inner lumen of the vessel 32, as shown in FIGS. 7 and 8. Alternatively, the compressed body 1 may be maintained in the compressed configuration with the tether 23 until the compressed body 1 is fully inserted into the vessel 32, and only then is the tether released to allow the body 1 to expand.

Once the body 1 is fully expanded, as shown in FIGS. 7 and 8, the body 1 is circumferentially apposed with the vessel wall 31, while distributing radial force outwards (as represented by arrows), which anchors the body 1 in place in the vessel 32. Additionally, the hub 4 and neck 2 are located in or extend outward from the vessel aperture 30. Once deployed, the delivery system can be detached (e.g., by rotation of the shaft 22) and the implant 100 left anchored in position. The hub 4 and neck 2 remains within the vessel aperture and is sealed by the occlusive material 10 of the implant 100.

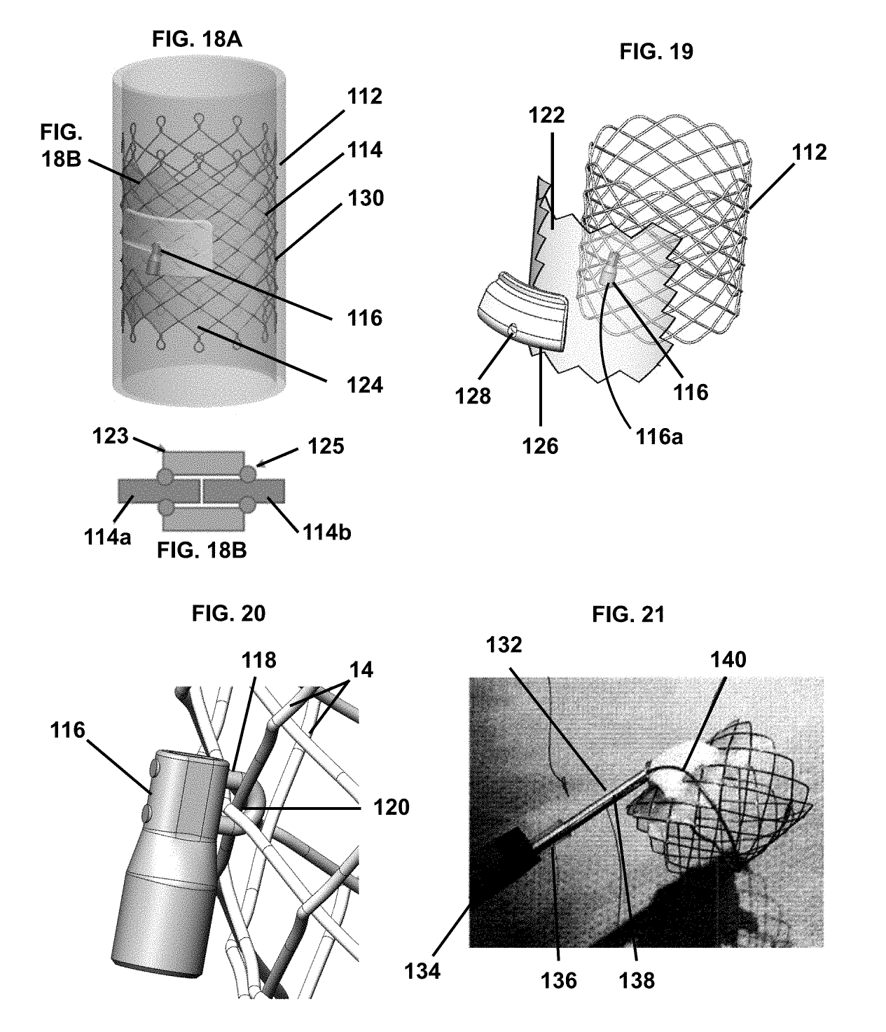

FIGS. 18A through 20 illustrate another embodiment of an implant 110. The implant 110 includes a tubular body 112 formed from a tubular mesh 114 that is biased radially outwardly and generates radial force when compressed. The mesh 114 may be formed from an elastic shape memory material and is able to undergo compression and expansion. The mesh 114 may be formed from a single wire, preferably with the free ends of all wire or wires terminating in crimped connections, such as is shown in FIGS. 18A and in greater detail in FIG. 18B. As shown in FIG. 18B, free ends 114a and 114b of wire forming the mesh 114 are secured together by a crimped circular tube 123 and circumferential welds 125. The circular tube 123 is crimped to both ends 114a and 114b and circumferential welds 125 are applied between end 114a and one end of tube 123 and welds 125 are applied between end 114b and an opposite end of tube 123.

The hub 116 is configured to connect to any control systems used for the manipulation and deployment of the implant 110. The hub 116 is movably (e.g., pivotally) coupled to the tubular mesh 114 with an eyelet 118 (FIG. 20) loosely extending about an intersection or crossing 120 of the tubular mesh wire 114.

As shown in FIGS. 18 and 19, an occlusive urethane coating 122 extends over cells 124 of the mesh 114 about a region of the side of the mesh 114 surrounding the location where the hub 116 is connected to the mesh 114. The coating 122 may be coated on the outside of the tubular mesh 114, on the inside of the tubular mesh 114, and/or within the interstices of the cells 124 of the mesh 114. A sealing material 126 in the form a fabric or other suitable materials is provided over the hub 116 and surrounding regions on the tubular mesh 114. The sealing material 126 preferably covers a smaller surface area than the coating 122. The sealing material 126 includes an opening 128 (FIG. 19) through which a proximal end 116a (FIG. 19) of the hub 116 is configured to extend. As shown most clearly in FIG. 18, the tubular mesh 114 defines tether loops 130 or openings (which may be no more than the open cells 124 within the mesh 114).

FIG. 21 shows a deployment device 132 coupled to the implant 110. The deployment device 132 includes a delivery tube or catheter 134, a deployment catheter 136, an actuation shaft 138, and a tether 140 extending about the tubular mesh 114. The deployment device 132 is configured to support the implant in a radially collapsed configuration. The tether 140 has two free ends 142, 144 (FIG. 24) that extend back through the delivery tube 134.

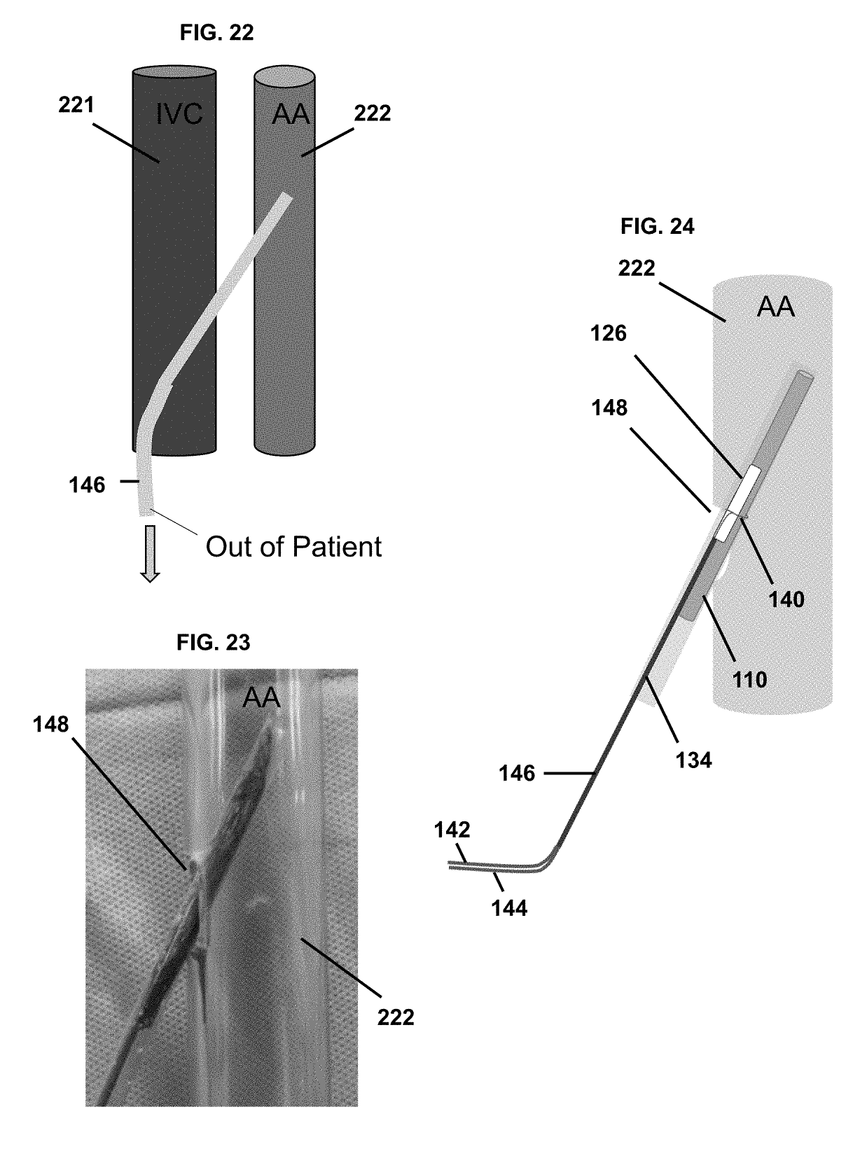

The implant 110 and deployment device 132 are configured for use in a transcaval procedure, illustrated in FIGS. 22, 23, 24, and 25a-25f Turning first to FIG. 22, a guidewire 146 can be first inserted through the inferior vena cava (IVC) 221 and extended in a crossing manner through the wall of the IVC and into the abdominal aorta (AA) 222. An instrument (not shown) is passed over the guidewire 146 to perform a procedure up through the AA 222. After the procedure, the instrument (not shown) is removed, leaving a surgical aperture 148 (FIG. 23) in the wall of the AA 222 that must be sealed.

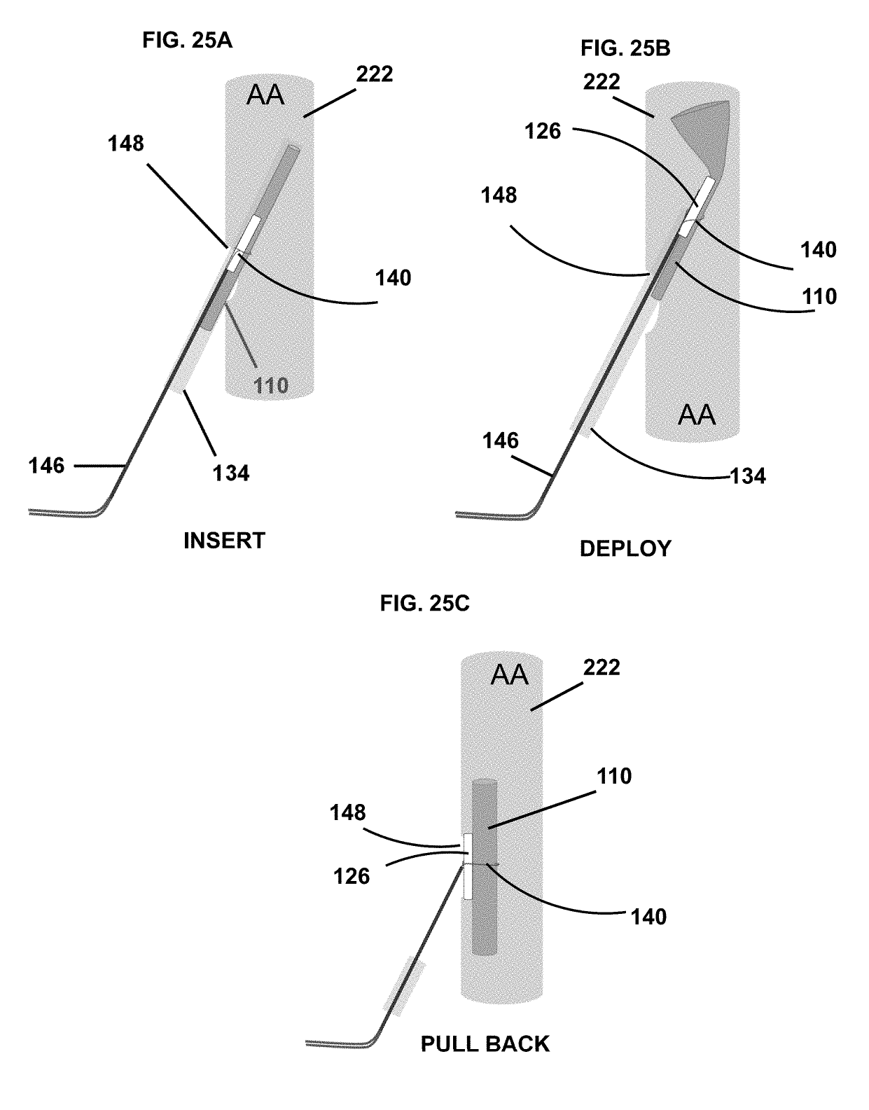

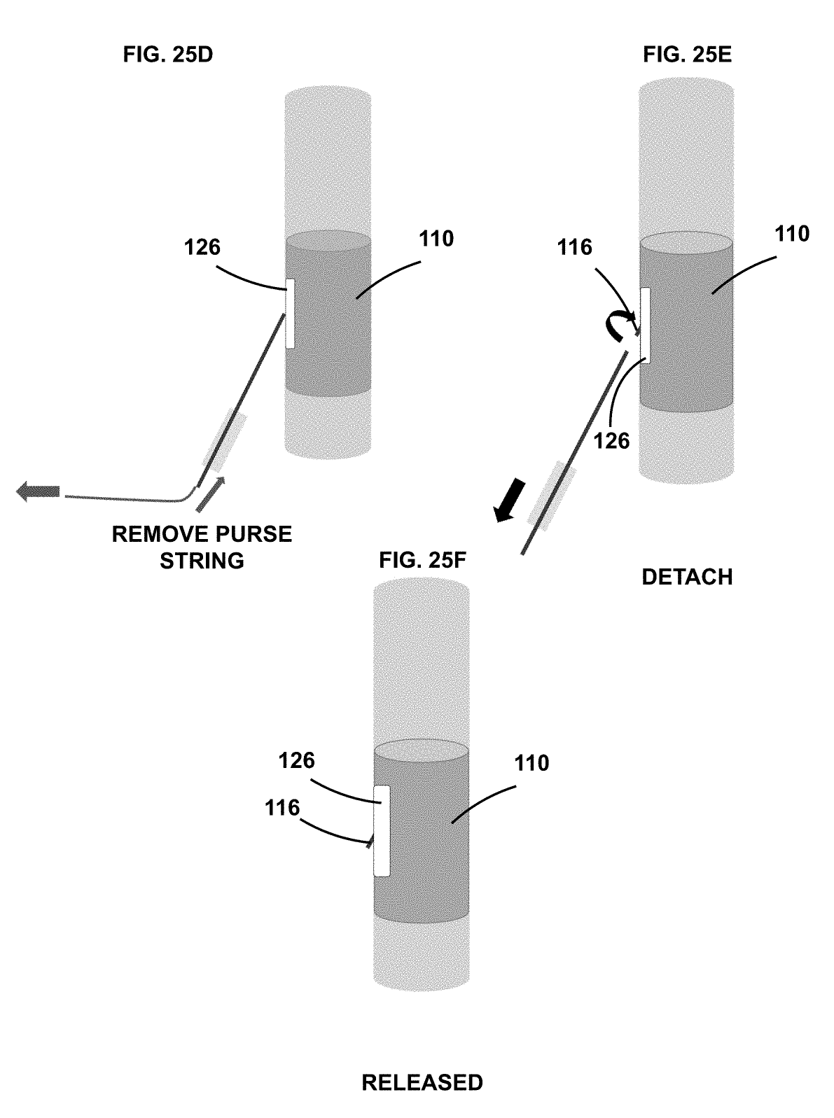

With reference to FIGS. 24 and 25a-f, one approach is as follows. The delivery tube 134 is advanced over the guidewire 146 through the IVC 221 and to the aperture 148 (FIGS. 24 and 25(a)). The delivery catheter 134 contains at its distal end the surgical implant 110 in a collapsed and tethered configured. The actuation shaft 138 (FIG. 21) of the deployment device 132 (FIG. 21) is actuated to advance the surgical implant 110 out of the distal end of the delivery catheter 134 and into the AA 222, and the delivery catheter 134 is retracted (FIG. 25(b)). Then, a slight proximally directed force can be applied to the surgical implant 110 until resistance is perceived by the user actuating the shaft 138, which indicates that the hub 116 is aligned with the transcaval aperture 148 in the AA 222 (FIG. 25(c)). Thereafter, one end of the tether 140 can be released while another end of the tether 140 is retracted to draw the tether 140 out of the delivery tube 134 and permit the surgical implant 110 to expand against the inside wall of the AA 222 (FIG. 25(d)). The actuation shaft 138 can then be rotated or otherwise operated to release the hub 116 from a distal end of the shaft 138 (FIGS. 25(e)-(f)). The deployment device 132 can then be withdrawn back into the IVC 221 and removed from the patient.

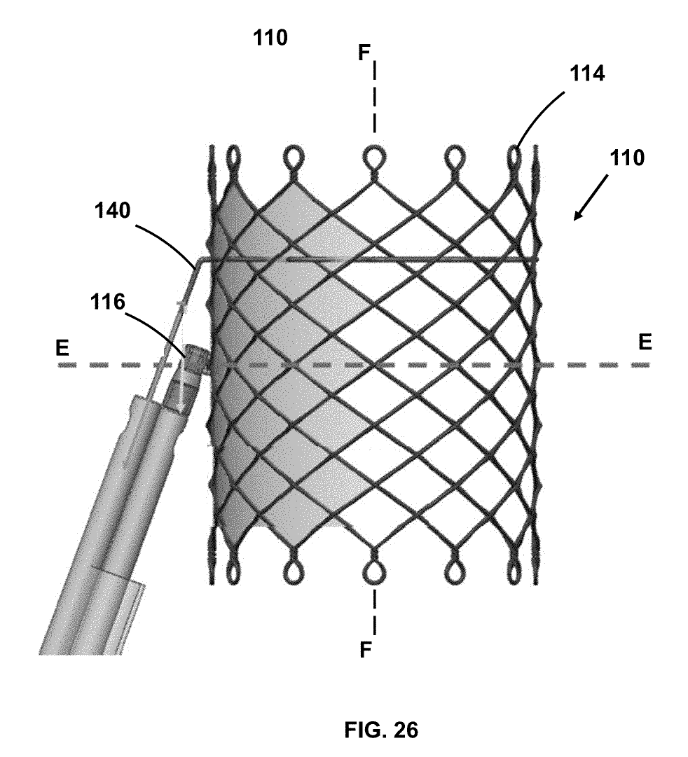

FIG. 26 illustrates an alternative approach to tethering the implant 110 above a midpoint E-E of the mesh body 114. In FIG. 26, the tether circumferentially surrounds the expanded implant 110 at a longitudinally spaced (measured along central axis E-E) position from that of the hub, which is located at the midpoint E-E. The hub 116 acts as a fulcrum about which the expanded mesh 114 can be rotated based upon tension applied to the tether 140. Thus, if a user increases tension in the tether 140, the force will urge the expanded mesh body 114 to rotate counter-clockwise (in FIG. 26) about hub 116, whereas reducing the tension in the tether 140 will tend to urge rotation in a clockwise direction. Thus, tension in the tether 140 can be used to control the rotational bias of the mesh body 114 about the hub 116 as the body expands upon its introduction into a vessel and/or after the body has fully expanded in the vessel.

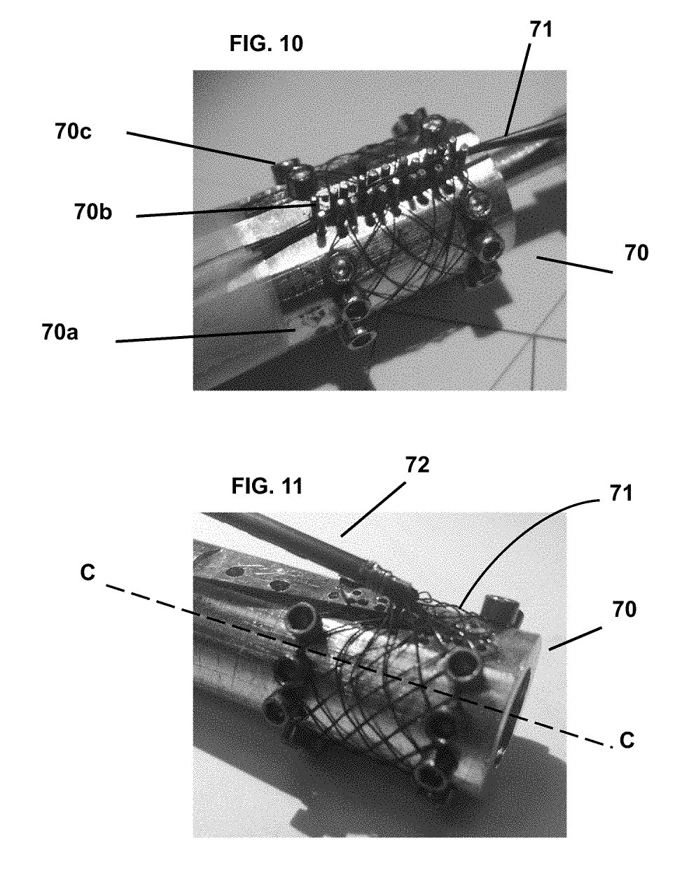

FIG. 10 shows a forming fixture 70 for shape setting a woven nitinol tubular structure where free ends of nitinol wires 71 are arranged in an opposing fashion. The forming fixture 70 includes a rod 70a with a plurality of radially extending pins 70b and screws 70c to allow the interweaving and fixation of the nitinol wires 71 in a specific pattern. FIG. 11 shows the nitinol wires 71 being joined together by a tube 72 extending at a non-zero angle with respect to the fixture's central axis C-C. The fixture 70 and nitinol wires 71 are heated and then quenched to yield a shape-set structure having a tubular form.

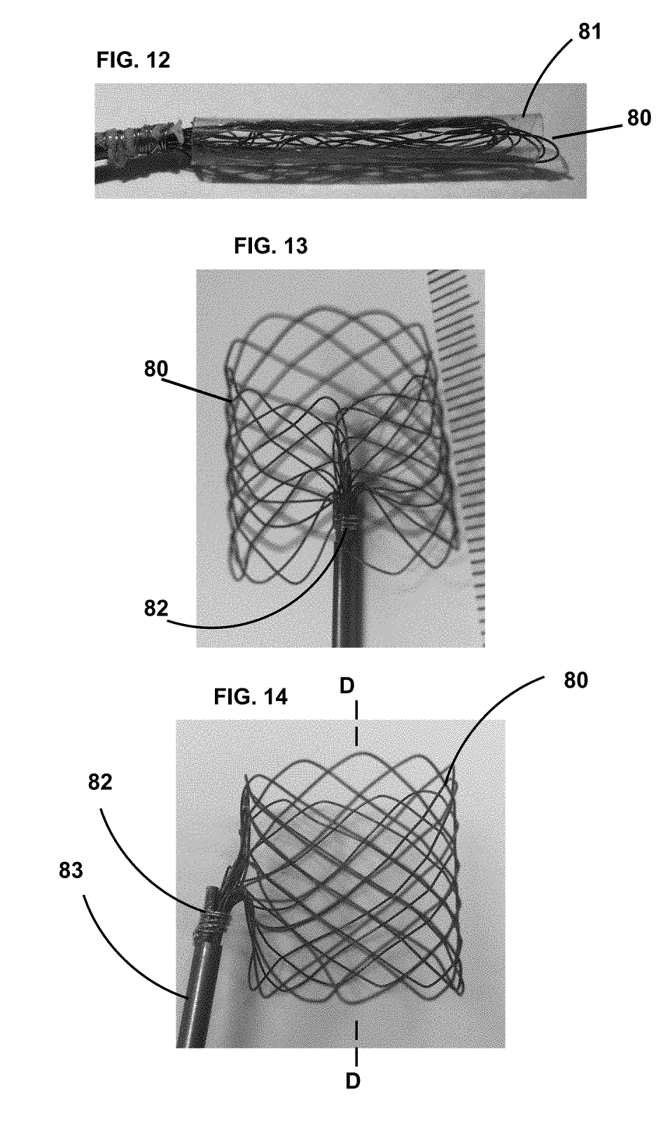

FIG. 12 illustrates a collapsed tubular, shape-set woven structure 80 in a collapsed configuration and constrained within a tube 81. FIG. 13 illustrates the shape-set tubular woven structure 80 in an expanded configuration with free wire ends of the woven structure 80 extending from the tubular structure to a central hub 82. FIG. 14 shows a side view of the woven structure 80 of FIG. 13 where the central hub 82 is connected to a connection member 83 that extends at a non-zero angle with respect to a central axis D-D of the woven structure 80.

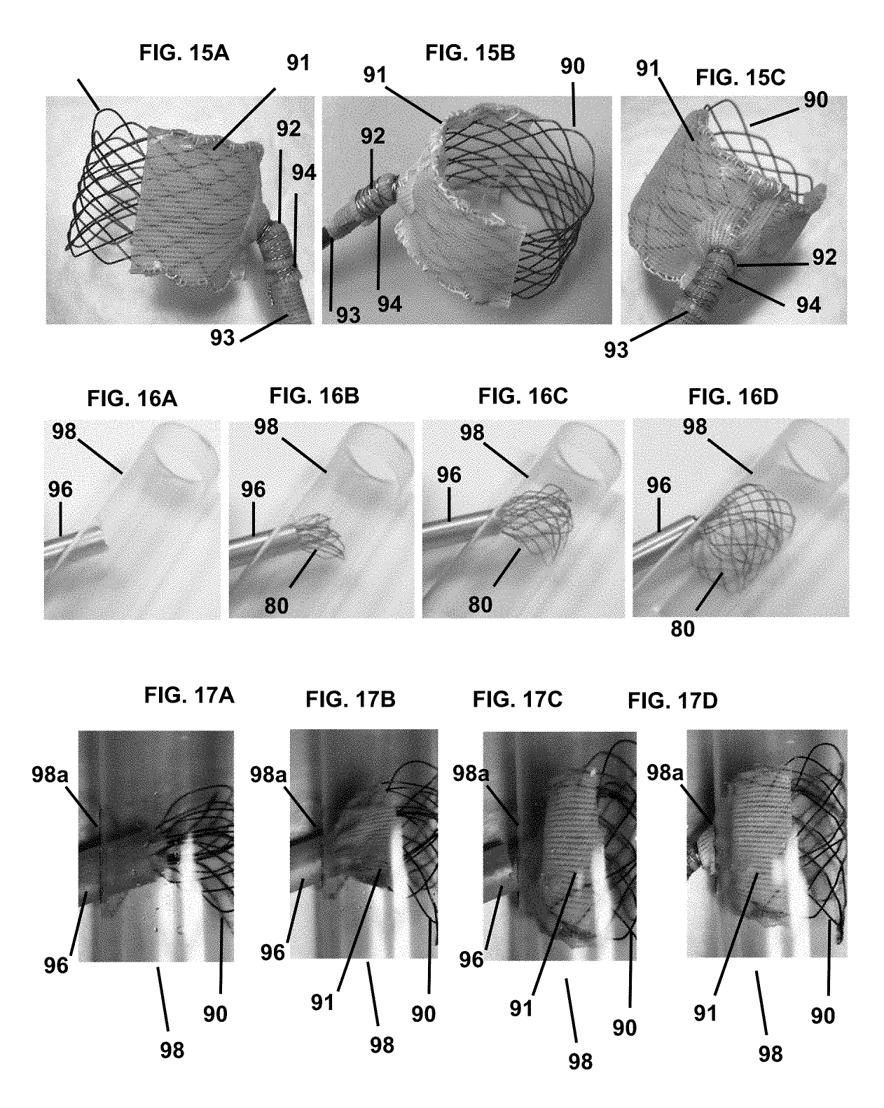

FIGS. 15A-15C another a tubular woven structure 90, constructed like structure 80, with a partial section covered by a hemostatic fabric 91. The fabric conforms to the structure up to the central connection member 92. At the central connection member 92, the hemostatic fabric 91 is disposed between a connection member 93 and strands 94 of the woven structure 90 extending outward form the side of the woven structure 90.

FIGS. 16A-16D illustrate sequential stages of deployment of the tubular woven structure 80 from a delivery tube 96 into a clear tube 98, which is representative of a vessel such as the AA 222.

FIGS. 17A-17D illustrate sequential stages of deployment of the tubular woven structure 90 from the delivery tube with the sealing member 91 into the clear tube 98. Prior to deployment the clear tube will leak through its side opening if it's pressurized with fluid. After deployment, the sealing material 91 seals an opening 98a and is held in place by the tubular structure 90. Also, after deployment, the central connection member 92 extends outwardly through the opening 98a from the side of the tube 98.

There have been described and illustrated herein several embodiments of an implant and a method of using the implant. While particular embodiments of the invention have been described, it is not intended that the invention be limited thereto, as it is intended that the invention be as broad in scope as the art will allow and that the specification be read likewise. Thus, while particular configurations of the body of the implant have been disclosed, it will be appreciated that other structures having similar properties to those disclose may be used as well. In addition, while particular types of materials have been disclosed, it will be understood that other materials having similar properties to those disclosed can be used. It will therefore be appreciated by those skilled in the art that yet other modifications could be made to the provided invention without deviating from its spirit and scope as claimed.

1. An implant comprising:
a collapsible tubular body, which, in an expanded configuration, extends from a first end to a second end centered along a longitudinal axis; and
a hub flexibly coupled to the tubular body between the first and second ends, the hub configured to removably connect to a deployment device.

2. The implant of claim 1, wherein:
the hub is pivotally coupled to the tubular body.

3. The implant of claim 1, wherein:
the tubular body is formed as an elastic mesh having a plurality of spaced cells.

4. The implant of claim 1, wherein:
the mesh is comprised of one or more fibers terminating at the hub.

5. The implant of claim 1, further comprising:
a tether coupled to the implant, the tether having a first configuration surrounding the body to retain the implant in a collapsed configuration and having a second configuration not surrounding the body to release the body from the collapsed configuration and permit the body to expand to the expanded configuration.

6. The implant of claim 5, wherein:
the body includes attachment points for coupling to the tether.

7. The implant of claim 1, wherein:
the body is biased into an expanded configuration and is configured to be compressed into a elongated collapsed delivery configuration for delivery into an opening in a vessel of a patient.

8. The implant of claim 7, wherein:
the implant is configured for insertion into a delivery catheter or tube when the body is compressed into the elongated delivery configuration.

9. The implant of claim 1, further comprising:
a hemostatic or occlusive material covering a portion of the tubular body at least adjacent the hub, the hemostatic or occlusive material configured to create a hemostatic seal about an opening in a vessel wall.

10. The implant of claim 9, wherein:
the occlusive material includes at least one of a sealing fabric and a coating.

11. The implant of claim 1, wherein:
the portion of the tubular body covered by the hemostatic or occlusive material is hemi-cylindrical and centered about the hub.

12. The implant of claim 1, wherein:
the body is formed of a shape-set memory material.

13. The implant of claim 1, further comprising a neck extending outwardly from a side of the tubular body to the hub, the neck extending at a non-zero angle with respect to the longitudinal axis.

14. An implant kit comprising:
a tube; and
an implant packaged in the tube and configured in a collapsed configuration, the implant including:
a collapsible tubular body, which, in an expanded configuration, extends from a first end to a second end centered along a longitudinal axis, the body being collapsed when packaged in the tube, and
a hub coupled to the tubular body between the first and second ends, the hub configured to removably connect to a deployment device; and
a deployment device coupled to the hub.

15. The kit of claim 14, further comprising a tether surrounding the body in the tube, the tether having at least one end extending through the tube and accessible by a user.

16. The kit of claim 14, wherein the deployment device includes an elongated shaft.

17. The kit of claim 16, wherein a connection between the shaft and the hub is a threaded or a bayonet connection.

18. The kit of claim 14, wherein when the implant is packaged in the tube, the central longitudinal axis of the implant is perpendicular to a central longitudinal axis of the tube.

19. A method of implanting an implant comprising:
(i) providing an implant kit comprising:
a tube; and
an implant packaged in the tube and configured in a collapsed configuration, the implant including:
a collapsible tubular body, which, in an expanded configuration, extends from a first end to a second end centered along a longitudinal axis, the body being collapsed when packaged in the tube, and
a hub coupled to the tubular body between the first and second ends, the hub configured to removably connect to a deployment device; and
a deployment device coupled to the hub;
(ii) introducing a distal end of the tube into an opening in a vessel;
(iii) translating the hub in the tube towards the distal end of the tube to introduce the collapsible tubular body into the vessel, whereupon its introduction, the tubular body coaxially self-aligns with a lumen of the vessel;
(iv) uncoupling the deployment device from the hub; and
(v) withdrawing the deployment device and the tube from the vessel so that at least the hub extends in or outward from the opening in the vessel.

20. The method of claim 19, wherein:
the implant further includes a tether coupled to the implant, the tether having a first configuration surrounding the body to retain the implant in a collapsed configuration and having a second configuration not surrounding the body to release the body from the collapsed configuration and permit the body to expand to the expanded configuration; and
wherein the method further includes:
(vi) configuring the tether to its second configuration to permit the body to expand to the expanded configuration upon its introduction into the vessel.

21. The method according to claim 19, wherein:
the implant further includes a hemostatic or occlusive material covering a portion of the tubular body at least adjacent the hub, the hemostatic or occlusive material configured to create a hemostatic seal about an opening in a vessel wall upon withdrawing the deployment device and the tube from the vessel in (v).
